(19) United States
(12) Patent Application Publication
(10) Pub. No.: US 2007/0197969 A1
(43) Pub. Date: Aug. 23, 2007

(54) Device for pressurization

(75) Inventors: Olle Hillborg, Solna (SE); Stefan Persson, Akersberga (SE)

Correspondence Address: BROOKS KUSHMAN P.C., 1000 TOWN CENTER, TWENTY-SECOND FLOOR, SOUTHFIELD, MI, 48075, US

(73) Assignee: QF MEDTECH AB, Stockholm (SE)

(21) Appl. No.: 11/571,163

(22) PCT Filed: Jun. 1, 2005
(86) PCT No.: PCT/SE05/00830

§ 371 Date: Dec. 22, 2006

(30) Foreign Application Priority Data
Jun. 24, 2004 (SE) 0401634-1

(51) Int. Cl. A61M 37/00

(52) U.S. Cl. 604/142

(57) ABSTRACT

An arrangement for pressurizing an inflatable sleeve (2) that can be applied, for instance, to the arm of a patient for measuring the patient's blood pressure, or that can be applied to an infusion bag for forced infusion of said infusion to a patient, wherein the device further includes a sleeve-connected pressure gauge (3), a hand pump (30) which is connected to the sleeve (2) via line (54,2) and which includes an air inlet (31) that has a check valve (32), wherein the device also comprises a manually actuable air release valve (55) for controlled release of gas from the sleeve interior, wherein a box (5) is connected in the line (52,4) between the sleeve (2) and the air release valve (53); wherein the box (5) is connectable to a pressurized gas source (20) by means of a hose (8), wherein the hose (8) connects with the interior of the box (5) via a check valve (7) and a manually controllable valve (6) for the input of gas from the pressure gas source to the sleeve (2) via the box (5).

The present invention relates to an object pressurizing device of the kind defined in the preamble of claim 1.

U.S. Pat. No. 4,088,126 teaches a device of the kind defined in the preamble of claim 1 for measuring blood pressure, wherein the device includes an inflatable sleeve for restricting the flow of blood in the arm of a patient for instance, wherein the sleeve is connected to a pressure gauge, and wherein a hand pump is connected to the sleeve via a line and includes an air inlet having a check valve, wherein the device also includes a manually actuable air release valve for controlled release of air from the sleeve. According to U.S. Pat. No. 4,088,126, a compressed air pump driven by an electric motor can be connected to the air inlet of the hand pump. The electric motor is connected to the mains network and includes a control means which can be maneuvered with the foot of an operator.

In a hospital or medical care environment it is normally required that the device can be moved readily between different places and that it can be readily used at said places. The requirement of access to an electric socket connected to the mains network and the transportation difficulties associated with an electrically driven compressed air pump with associated foot switch limits the usefulness of this known device.

Accordingly, an object of the present invention is to provide a device in which the sleeve can be alternatively inflated from a compressed gas network that is normally found in a hospital or care environment and which normally has a compressed air outlet available at a large number of places in the wards and rooms of the hospital.

This object is achieved by the invention.

The invention is defined in the accompanying claim 1.

Further embodiments of the invention are made apparent in the accompanying dependent claims.

The invention enables the user to use the hand pump to inflate the sleeve, in a conventional manner. When a compressed gas outlet is available, the user can readily connect the plug to the pressurizing gas outlet and control inflation of the sleeve through the medium of a corresponding manually actuable valve on the box. Even though the box is connected to the pressure gas outlet, the user is able to use the hand pump to pressurize the sleeve, if he/she so desires.

In addition to being used to measure the blood pressure of a patient, the inventive device can also be used to pressurize a flexible container, for instance a bag containing an infusion (infusion solution and/or blood) which shall be infused into a patient from the bag at an overpressure that can be set with the device, for instance via a hose connected to the bag and provided with a cannula that is inserted into a blood vessel of the patient.

The pressure gas source may, alternatively, consist in a pressure gas bottle. Such pressure gas bottles are often carried on boats, for instance patient transporting vehicles, thereby enabling an inventive device to be used on boats or in vehicles.

Particularly preferred embodiments of the inventive device will now be described by way of example with reference to the accompanying drawing, in which

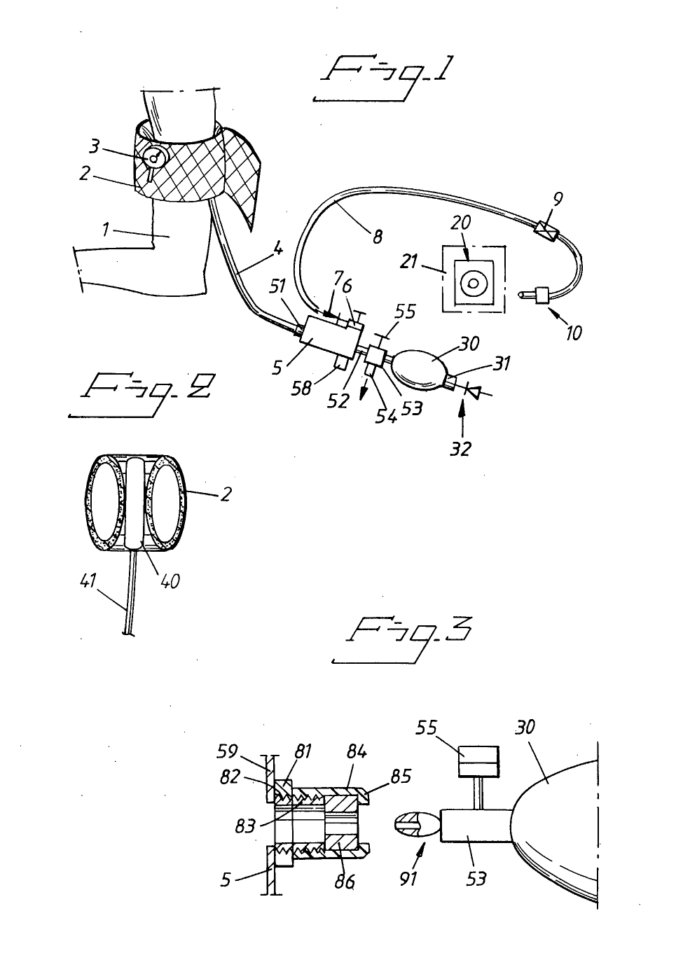

FIG. 1 is a diagrammatic illustration of a device according to the present invention applied to the upper arm of a patient with the intention of measuring his/her blood pressure;

FIG. 2 illustrates the device applied to an infusion bag whose content shall be infused into a patient; and

FIG. 3 illustrates a connection between a box and a hand pump included in the device.

An inflatable sleeve 2 is shown applied around the patient's arm 1 and connected to a pressure gauge 3. A hand-operated balloon-type air pump 30 includes an air inlet opening 31 which includes a check valve 32. The hand pump 30 is connected to the interior of the sleeve 2 for inflating said sleeve, via a line 32, a box 5 and a hose 4, so that the sleeve is able to restrict the flow of blood in the arm 1. An air release valve 33 is connected to the outlet line 52 of the hand pump 30 and includes a manually actuable element 55 which, when activated allows air to be released from the interior of the sleeve 2 through an outlet 55, via the line 4, the box 5 and the line 54.

The box 5 has an outlet connection 51 to which the hose 4 is connected. The box 5 also includes a manually controllable inlet valve 6 for the input of compressed air from a pressure gas source to the interior of the box 5. The pressure gas source consists in a pressure gas network that has been installed in a hospital environment and which has an accessible socket outlet 20 mounted on a wall 21, such outlet sockets being found in many rooms of the hospital. A hose 80 is provided with a connector 10 that can be plugged into the wall socket 20. The hose 8 connects to the valve 6. As shown, the hose 8 includes a restriction for setting an upper limit at which the flow of gas can be passed to the interior of the box 5. The hose 8 is shown connected to the valve 6 via a check valve 7. It will be understood by the person of average skill in this art that the restriction valve and the check valve 7 may be given other placements in the flow path between the wall outlet 20 and the interior of the box 5, although the preferred placement is on the box.

The box may include an emergency valve 50 which functions to release air automatically from the box 5 should the pressure in the box exceed a pre-determined value. The flow through the restriction 9 will, of course, be lower than the flow that can depart through the emergency or safety valve 50.

The connecting piece 10 on the hose 8 is conveniently adapted for co-action with the wall mounted outlet 20 so as to hold a closure valve open when in engagement with said outlet 20. The hose 8 may be realisably connected to the check valve 8. The person of average skill in this art will realize that the input valve 6 and the check valve 7 can swap places in the air inflow path to the box 5. The pressure gas source 20 is normally a terminal to a pressurized breathing gas network. Alternatively, the pressure gas source may comprise a pressure gas bottle. Such pressure gas bottles are often carried in vehicles and on boats, particularly in ambulances and other patient transporting vehicles, therewith enabling the inventive device to be used also in such vehicles and boats.

FIG. 2 illustrates the inflatable sleeve 2 surrounding a bag 40 of infusion solution, which can thereby be infused into a patient at a settable overpressure, which may be desirable in certain instance in order to maintain or to strengthen the infusion supply, for instance, via the hose 41 connected to the bag 40 and for instance via a cannula (not shown) connected to the hose and receivable in a blood vessel of the patient.

FIG. 3 illustrates a valve 53 which is connected to the pumping balloon 30 and which includes an elongate, externally oval and longitudinally projecting tubular part 91. The wall of the box 5 includes a channel in which a tubular rubber bush 86 is clamped. The through-flow channel of the bush 86 is smaller than the maximum diameter of the tubular part 91 and serves to accommodate said part, wherewith the change in the shape of the bush 86 can be taken up by the ability of the rubber bush to deform elastically. As will be seen from FIG. 3, the bush 86 is tensioned axially in an internal groove that extends circumferentially in a tubular stud which projects out from the wall of the box 5. It will be seen that a nut 81 is firmly connected to the outside of the wall 59 around a medium through-flow opening therein. An externally threaded bush 83 is firmly screwed into the nut 81 and supports at its free end against the rubber bush 86. A plastic sleeve 84 is placed externally on the bush 83 and projects beyond the bush in an axial direction and includes radially bent end edges 85. The rubber bush 86 is clamped axially between the flange 85 and the outer end of the bush 83. The part 91 is accommodated firmly in the rubber bush 86 although being releasable therefrom, and is able to swing to some extent in the bush while retaining a tight connection therewith. The part 91 can be readily pulled out of the bush 86 manually when necessary, and can be readily fitted to the bush 86.

In practice, the sleeve 84 together with the bush 86 can be placed on the inside of the box. In addition, the sleeve and its ring element that enclose the bush radially and axially may be produced in some other way, for instance by injection moulding or some technically equivalent method.

1. A device for pressurizing an inflatable sleeve, wherein the device comprises a pressure gauge connected to the sleeve, a hand pump which is connected to the sleeve via a line and which includes an air inlet having a check valve, wherein the device further comprises a manually actuable air release valve for the controlled release of gas from the sleeve interior, wherein a box is connected in the line between the sleeve and the air release valve Y(53); in that the box can be connected to a pressure gas source by means of a hose, wherein the hose connects with the interior of the box via a check valve and a manually controllable valve for the input of gas from the pressure gas source to the sleeve via the box; wherein the pressure gas source includes an outlet for a gas distribution system in a hospital, wherein the gas is preferably pressurized breathing air; and wherein the hose that can be connected to the pressure gas source includes a connecting piece that co-acts with the outlet such as to open a closure valve therein and therewith permit pressurized gas to flow through the hose.

2. A device according to claim 1, wherein a restriction for limiting the flow of gas from the pressure gas source is connected in the flow path between the pressure gas source and the interior of the box.

3. A device according to claim 1, wherein an overpressure valve for limiting the interior gas pressure of the device to a pre-determined value in an upward sense.

4. A device according to claim 1, wherein the valve for the input of gas from the pressure gas source is biased towards a closed state.

5. A device according to claim 1, wherein the box-associated check valve is mounted in the box.

6. A device according to claim 1, wherein the pressure gas source is a pressure gas bottle that preferably contains pressurized breathing air and that can be transported by a patient transporting vehicle.

7. A device according to claim 1, wherein the sleeve is adapted for application to the arm of a patient in order to measure the patient's blood pressure, or in that the sleeve is intended for application to a bag that contains infusion solution and/or blood, for pressurizing the same and diffusing the solution and/or blood into a patient.

8. A device according to claim 1, wherein the box includes an inlet channel in which a tubular rubber brush is clamped; and in that the hand pump includes an outlet line having an outwardly projecting oval line-part which can be forced into the through-flow channel of the rubber brush for releasable and conductible attachment.
